(19) United States
(12) Patent Application Publication
(10) Pub. No.: US 2019/0133898 A1
(43) Pub. Date: May 9, 2019

(54) METHOD FOR PREPARING AN EMULSION

(71) Applicants: INSTITUT POLYTECHNIQUE DE GRENOBLE, Grenoble (FR); PIERRE FABRE DERMO-COSMETIQUE, Boulogne (FR)

(72) Inventors: Julien BRAS, Saint Martin D'Uriage (FR); Bernard FABRE, Boulogne (FR)

(21) Appl. No.: 16/094,787

(22) PCT Filed: Apr. 12, 2017
(86) PCT No.: PCT/FR2017/050882

§ 371 Date: Oct. 18, 2018

(30) Foreign Application Priority Data
Apr. 18, 2016 (FR) 1653400

(51) Int. Cl. A61K 8/06; A61K 8/73; A61Q 5/00; A61Q 19/00

(52) U.S. Cl. CPC A61K 8/062; A61Q 19/00; A61Q 5/00; A61K 8/731

(57) ABSTRACT

The present invention relates to a method for preparing dermo-cosmetic compositions in the form of emulsions, which as emulsifier contain cationized cellulose fibres with a degree of cationic substitution greater than 0.15, the concentration of the microfibers being great than 0.8% by dry weight in relation to the total weight of the composition. The invention further relates to the use of said cationized cellulose fibres as emulsifying agent, and to the emulsions prepared using said method.

The present patent application claims the priority of the French patent application FR16/53400 which will be considered as forming an integral part of the present description.

### FIELD OF THE INVENTION

The present application relates to a method for preparing dermo-cosmetic compositions in the form of emulsions, by using cationised cellulose fibres as an emulsifier, as well as emulsions prepared from this method.

### DESCRIPTION OF THE PRIOR ART

In numerous industries, such as cosmetics, food, the paint industry, the latex coatings industry (on paper, textile, plastic film), the transformation of elastomer plastic materials, use emulsions with a very high tonnage.

The search for new, non-toxic solutions, and solutions of biological origin is very active to meet clients' expectations (particularly in cosmetics). These solutions of biological origin are today rare and expensive, even if a few surfactants of biological origin are arriving on the market. However, the legalisation or stability of the systems limits the use thereof today. Proposing new solutions has a dual interest—both economic and environmental.

Microfibrillated Cellulose (MFC) is obtained in the form of an aqueous suspension by enzymatic pre-treatment and mechanical treatment. The interest from manufacturers for this material is growing, in particular following the announcement from the industrialisation thereof, but also following the numerous properties they give: mechanical reinforcement, nano-network barriers, salting-out system, rheology modifier, ink additives, etc.

In 2014-2015, the first publications have been proposed to make emulsifying suspensions with these elements in very low quantities. Very recently, new research is attempting to make anionic or cationic cellulose microfibrils from different sources. Nobody has tested them in emulsion, to date. Nobody has tested preparing microfibrils at the same time as emulsion, either, i.e. making a fibrillation of fibres during the method of preparing a dermatological or cosmetic composition (this method will be called in situ fibrillation).

There is a need for innovative methods of preparing dermatological or cosmetic compositions, and in particular dermo-cosmetic, homogenous and stable emulsions, over time.

There is a need for such dermo-cosmetic compositions obtained exclusively from agro-sourced products.

### BRIEF DESCRIPTION OF THE DRAWINGS

The characteristics and advantages of specific embodiments, as well as others, will be described in detail in the following description, made in a non-limiting manner, in relation to the attached figures, among which:

FIG. 1 represents an embodiment of a method for obtaining a dermo-cosmetic composition in the form of emulsion by using fibrillated cellulose fibres during the method as an emulsifier;

FIG. 2 represents the comparison in terms of stability of the two emulsions prepared from non-cationised or cationised cellulose microfibres used as an emulsifier; and

FIG. 3 represents the creaming index of emulsions according to the concentration of cationised cellulose fibres, the emulsion having been prepared according to the method of FIG. 1.

### DETAILED DESCRIPTION

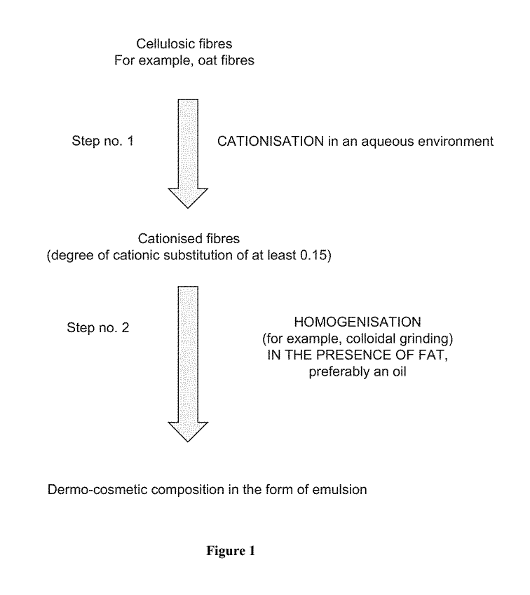

FIG. 1 illustrates an embodiment of a method for obtaining a stable suspension of fibrillated cationic fibres during, and up to, the obtaining of a dermo-cosmetic composition in the form of emulsifying suspension or stable emulsion. In this case, in situ fibrillation will be referred to.

By in situ fibrillation, this means the fibrillation of cationic fibres at the same time as obtaining a dermo-cosmetic composition, i.e. the passing through homogenisation of a fibre mixture in water with oil to a water, oil and fibrillated fibre (otherwise called microfibre)-based emulsifying suspension.

By emulsion, this means a close mixture of two liquid substances. These are always two liquids which, in a normal situation, are non-miscible but will, through specific operations, succeed in having a macroscopically homogenous, but microscopically heterogenous appearance. This mixture remains stable, thanks to a third ingredient called emulsifier.

By emulsifier, this means a chemical or natural product used to increase the smoothness of certain products, to stabilise an emulsion.

According to this method, cellulosic fibres are the starting point. These cellulosic fibres can advantageously come from parts, hardly or not at all harvested from plant cultures. The use of cellulosic fibres of fresh or dry plants is also possible. These “plant” group is formed from two lines, algae and plants. By plant, this means all or part of a plant, said plant being dry or fresh, frozen or unfrozen and whole, fragmented or ground. By “part of plant”, this means above-ground parts, such as stems, branches, leaves, seeds and/or flowers and/or underground parts such as rhizomes, roots and/or bulbs. Among useable and preferred plants, among others, oat can be cited, in particular from the above-ground parts, or oat seed coat, flaxseed, straw, sugarcane, even leek or eucalyptus wood. Advantageously, oat is preferred for the present invention.

At step 1, cellulosic fibres in an aqueous environment are subjected to a cationisation up to obtaining a degree of cationic substitution greater than 0.15. The degree of substitution is defined for a cellulosic fibre as being the ratio between the number of modified OH groups and the total number of OH groups per anhydroglucose unit, the cationisation step is well known to a person skilled in the art. This step is carried out in an exclusively aqueous environment.

At step 2, a homogenisation step is directly proceeded with, preferably by colloidal grinding, in the presence of at least one fat, this fat being preferably an oil. A person skilled in the art knows perfectly the conditions of using such homogenisation methods. A particularly homogenous and stable emulsion or emulsifying suspension is thus obtained directly (in situ) over time.

Preparing an emulsion requires an input of energy, a movement of fluids. Different mixing systems can be used for this present invention, in order to prepare emulsions. The mixture is preferably made in a mixer with a stirrer, constituted of a tank inside which is located the stirrer which can presented in different forms, bar magnet, turbine, rotor-stator device (ULTRA-TURRAX), etc. These stirrers can have more or less increased rotation speeds, going from a few hundred up to a few thousand rotations per minute. Another type of mixer can be used, particularly suitable are static mixers when the products are viscous. Finally, the emulsification can also be done by membrane. For example, at the laboratory scale, a mixer of the ULTRA TURRAX type has been selected, with the following conditions: at least 1 minute, with a rotation speed greater than 12000 rpm, with a volume of 100 ml and a sufficient shear stress greater than 10s⁻¹.

By homogenous emulsion, this means, in the sense of the present invention, an emulsion with a creaming index equal to zero. It will be reminded, that the creaming index is defined by placing the composition in a container and by measuring the ratio between the height of the non-emulsified part and the height of the emulsified part. Thus, a zero value of the creaming index corresponds to a perfectly homogenous and stable composition.

By stability, this means a good dispersion homogeneity of the internal phase, in the form of microdroplets, in the external phase. A stable composition is particularly a composition which undergoes no salting-out, dephasing, precipitation, coalescence, flocculation, creaming phenomenon, etc. The composition according to the invention is advantageously such that the balance of the phases which compose it is maintained, i.e. the dispersion of the internal phase in the external phase remains homogenous.

From such a preparation method, the composition according to the invention can furthermore advantageously be preserved for a long period, despite the absence of a preservative.

By “oil”, this means, in the sense of the present invention, a “fat” which is liquid at ambient temperature (25° C.), and at atmospheric pressure (760 mm Hg), solubility less than 5%, preferably 1%, also more preferably at 0.1%. Fats are generally soluble in organic solvents under the same temperature and pressure conditions, like for example chloroform, dichloromethane, ethanol, benzene, toluene or Vaseline oil.

More specifically, the oils are selected from among non-silicone oils, oils of animal origin, triglyceride oils of plant origin, synthetic fluorinated glycerides or oils, fatty alcohols, fatty acid esters and/or different triglyceride fatty alcohols, and silicone oils.

Among non-silicone oils, C₆-C₁₆, linear, ramified possibly cyclic alkanes can be cited. As an example, hexane, dodecane, isoparaffins like isohexadecane, isodecane can be cited. Linear or ramified hydrocarbons with more than 16 carbon atoms, can be selected from among paraffin oils, Vaseline, Vaseline oil, polydecenes and hydrogenated polyisobutene.

Among animal oils, perhydrosqualene can be cited.

Among triglycerides of plant or synthetic origin, liquid fat triglycerides comprising 6 to 30 carbon atoms, like heptanoic or octanoic acid triglycerides can be cited, or also, for example, sunflower, corn, soya, marrow oils, grape seed, sesame, hazelnut, apricot, macadamia, arara, castor, avocado oils, caprylic/capric acid triglycerides, jojoba oil, shea butter oil.

Among fluorinated oils, perfluoromethylcyclopentane and perfluoro-1,3-dimethyl-cyclohexane, perfluoro-1,2-dimethylcyclobutane, perfluoro-alkanes such as dodecalfluoropentane and tetra-decalfluorohexane, bromoperfluoroctyl, nonafluoro-methoxybutane, nonafluoroethoxyisobutane, perfluoromorpholine derivatives such as 4-trifluoromethyl perfluoromorpholine can be cited.

According to a most particularly preferred embodiment of the invention, the oil is selected from among oils of natural origin, more specifically oils of plant origin, preferably jojoba, safflower, babassu, sunflower, olive, coconut, Brazil nut, marula, corn, argan, soya, marrow, grape seed, flaxseed, sesame, hazelnut, apricot, macadamia, arara, coriander, castor, avocado oils, shea butter oil as well as colza oil.

Possibly, the fat can be different from oils, butters, waxes or resins can be cited.

The concentration of fat, the fatty phase of the composition according to the invention is referred to, represents between 5 and 70% by weight in relation to the total weight of the composition, preferably between 5 and 40% and also preferably, between 10 and 30%. Advantageously, the concentration of fat represents 10% by weight in relation to the total weight of the composition. Also advantageously, the fatty phase represents 30% by weight in relation to the total weight of the composition. More advantageously also, the fatty phase represents 30% by weight in relation to the total weight of the composition.

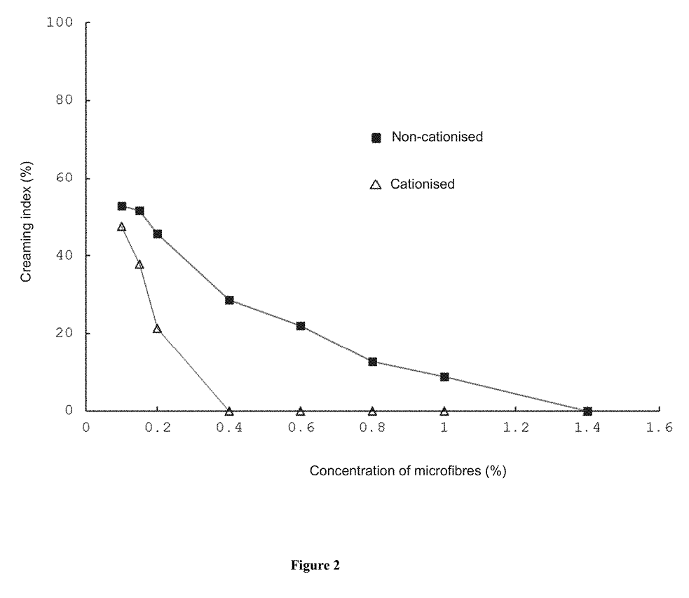

FIG. 2 is a comparative representation of the creaming index of two emulsions according to the concentration of cellulose microfibres. The two emulsions are prepared respectively with non-cationised and cationised cellulose microfibres, The method used in this example, is not that described above in FIG. 1. After the step of cationisation in an aqueous environment, the homogenisation is done without adding fat in order to obtain a cationised microfibre suspension. It is during a third step, that this suspension is mixed with at least one fat to obtain an emulsion. This emulsion contains 30% of oil and 70% of cationised microfibre aqueous suspension. The creaming index has been measured at 40 days, at ambient temperature, after the preparation of the emulsions.

It is observed that the emulsifying behaviour totally changes when the cellulose microfibres are cationised. Indeed, an emulsion is thus obtained with a creaming index near 0, even for very low concentrations of cellulose microfibres when they are cationised. Thus, a concentration of 0.4% of cationised cellulose microfibres is sufficient to obtain a perfectly homogenous and stable emulsion with a creaming index equal to 0 after 40 days. The results have been obtained with cationised cellulose fibres with a degree of substitution of 0.18. Generally, the result is obtained as soon as the degree of substitution is greater than 0.15. When the cellulose fibres are not cationised, three times more must be added to obtain a stable emulsion (FIG. 2). It must be noted, that no addition of bactericide is necessary to obtain a good preservation of the cosmetic composition according to the invention.

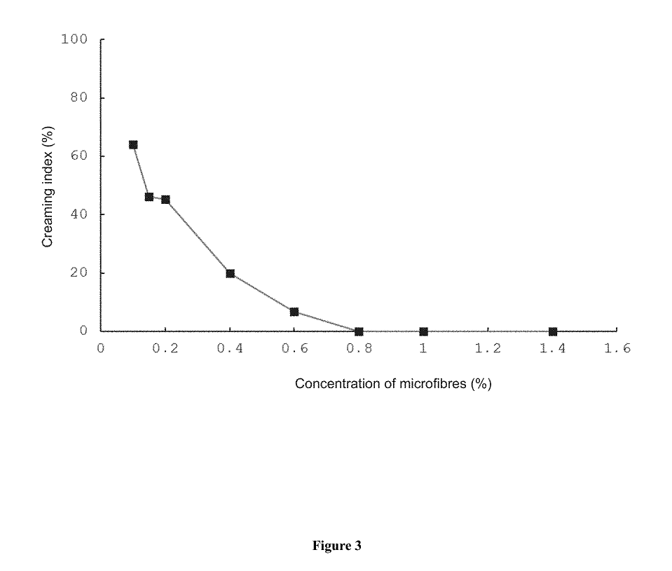

FIG. 3 represents the emulsion creaming index according to the concentration of cationised cellulose fibres. The method used to obtain emulsions is that described in FIG. 1 of in situ fibrillation during the obtaining of the emulsifying suspension. The emulsion contains 20% of oil and 80% of aqueous solution. The creaming index has been measured 60 days after the preparation of the emulsions, at ambient temperature.

It is observed that the creaming index decreases a lot when the concentration of fibres exceeds values greater than 0.2%. FIG. 3 clearly shows that it is from 0.8% of cationised cellulose fibres that the emulsion obtained is perfectly homogenous and stable, with a creaming index of 0.

This embodiment of emulsions is particularly useful at the industrial level, since it avoids any mixture step, usually present in this type of method. This embodiment therefore has the advantage of being particularly quick. As a guide, for batches of around 100 litres, in the case where a mixture step is necessary, a grinding of 2 hours must be followed by a mixture in the presence of fat, also of 2 hours. In the case of the method described in this present invention, a grinding in the presence of fat for only 2 hours is sufficient, highly limiting the energy necessary to produce the suspension. Another specificity of this method of production is that the concentration of cellulose microfibres in the emulsion can be relatively increased. Indeed, it can reach contents going up to 15%.

In conclusion, it has been shown that compositions in the form of emulsion, in particular for dermo-cosmetic use, can be obtained by using only cationised microfibres from above-ground parts or from the oat thereof as an emulsifier. The emulsifying properties thereof are very useful and the cosmetic qualities thereof (in particular, smoothness) have been proved. These compositions also have filmogenic and antibacterial properties—particularly useful in the dermo-cosmetic field.

Even if the inventors have perfectly proved that cationised cellulose microfibres were sufficient to obtain a homogenous and stable emulsion, according to a specific embodiment of the invention, the homogeneity and/or stability of the emulsion can sometimes also be improved by adding another emulsifying agent.

As examples, but not in a limiting manner, anionic surfactants such as stearates like sodium stearate, sodium cetostearyl sulphate, potassium cetyl phosphate; non-ionic surfactants such as polyoxyethylene (or not) sorbitan esters, macrogol esters, sucroesters, ethers, poloxamers, silicone derivatives, phospholipids can be cited.

By surfactant agent, this means, in the sense of the present invention, a compound which modifies the superficial tension between two surfaces.

According to a preferred embodiment of the invention, cationised cellulose microfibres are sufficient to obtain a homogenous and stable emulsion, no other emulsifying agent is added.

In the dermo-cosmetic composition according to the invention, the concentration of cationic cellulose fibres with a degree of cationic substitution of at least 0.15, is greater than 1.4% by dry weight in relation to the total weight of the composition. Preferably, this concentration is greater than 1.0% and also preferably, greater than 0.8% in relation to the total weight of the composition.

It has been shown that it was possible:

to obtain cationic microfibres in particular of a diameter greater than 100 nm and of a length greater than 1 μm from above-ground parts and the oat thereof after bleaching and pre-treatment;

to have stable suspensions (oil in water) with cationic cellulose microfibres if the degree of substitution thereof is greater than 0.15 and with a weighting percentage of microfibres in the solution greater than 0.8%;

to have cationic microfibre-based dermo-cosmetic compositions as an emulsifier, with optimal cosmetic qualities;

to favour an in situ fibrillation of cationic microfibres pre-treated during the method of mixing emulsions;

to obtain films of excellent quality after application on the skin.

The composition according to the invention can be prepared in the form of a simple oil in water or water in oil emulsion, a multiple emulsion like for example, a water emulsion in oil in water, or an oil emulsion in water in oil.

The composition according to the invention is configured for a cutaneous application. The dermatological or cosmetic composition according to the invention can further comprise, one or more usual excipients, which are dermatologically or cosmetically compatible.

Dermatologically or cosmetically compatible excipients can be any excipient among those known by a person skilled in the art with a view to obtain a composition for topical application in the form of a milk, a cream, a balm, a lotion, a gel, a foaming gel, an oil, an emulsion, a microemulsion, an ointment, or a spray. The composition according to the invention can furthermore contain additives and formulation aids, such as thickeners, gelling agents, water binders, spreading agents, stabilisers, colourants, perfumes, and preservatives.

To give specific properties to the composition, various active substances can be added to it, selected from among the group comprising perfumes, hydrating products, anti-wrinkle products, etc. These active substances can be included in the fat added to prepare the emulsion.

Thus, an embodiment provides a dermo-cosmetic composition in the form of emulsion, which as emulsifier contain cationised cellulose microfibres with a degree of cationic substitution greater than 0.15, the concentration of these microfibres being greater than 0.8% by dry weight in relation to the total weight of the composition.

Preferably, the cellulose microfibres come from oat, oat microfibres are referred to.

Advantageously, the emulsion thus prepared is a simple oil in water emulsion.

Advantageously, the fatty phase of the emulsion represents 10 to 30% by weight in relation to the total weight of the composition.

Preferably, the composition according to the invention does not contain any other emulsifying agent.

Another embodiment of the invention provides a use of a dermo-cosmetic composition in the form of emulsion which as emulsifier contains cationised cellulose microfibres with a degree of cationic substitution greater than 0.15, the concentration of these cationised microfibres being greater than 0.8% by dry weight in relation to the total weight of the composition, responding to one or more of the characteristics above, for cosmetic care by application topically on the skin, the mucosa and/or the hair.

To this end, the composition according to the invention can be applied on the skin, the mucosa and/or the hair topically.

Thus, a method for cosmetically treating the skin, the mucosa and/or the hair comprises the application topically, on the skin, the mucosa and/or the hair, of a composition according to the invention.

The present invention moreover relates to the use of a composition according to the invention for the curative and/or preventative treatment of diseases of the skin, the mucosa and/or the hair.

Another embodiment of the invention relates to a method for preparing a dermo-cosmetic composition in the form of emulsion, comprising the following steps:

proceeding with cationisation in an aqueous phase of cellulosic fibres up to reaching a degree of cationic substitution greater than 0.15, then

proceeding with a homogenisation of the cationised fibres in the presence of at least one fat up to obtaining a homogenous emulsion, the concentration of the cationised cellulose microfibres being greater than 0.8% by dry weight in relation to the total weight of the composition.

Preferably, this method is carried out with oat fibres.

Advantageously, the homogenisation step is done by colloidal grinding.

Advantageously, the fat used is an oil.

A last embodiment of the invention provides the use of cationised cellulose microfibres with a degree of cationic substitution greater than 0.15, as an emulsifier.

The use of cationised oat microfibres with a degree of cationic substitution greater than 0.15, as an emulsifier, is preferred.

1. A method for preparing a dermo-cosmetic composition in a form of emulsion, comprising the following steps:
proceeding with cationisation in an aqueous phase of cellulosic fibres up to reaching a degree of cationic substitution greater than 0.15, then
proceeding with a homogenisation of the cationised fibres in the presence of at least one fat up to obtaining a homogenous emulsion, the concentration of the cationised cellulose microfibres being greater than 0.8% by dry weight in relation to the total weight of the composition.

2. The method for preparing a dermo-cosmetic composition in the form of emulsion according to claim 1, wherein the cellulose fibres are oat fibres.

3. The method for preparing a dermo-cosmetic composition according to claim 1, wherein the homogenisation is done by colloidal grinding.

4. The method for preparing a dermo-cosmetic composition according to claim 1, wherein the fat is an oil.

5. A dermo-cosmetic composition in a form of emulsion, wherein it is obtained by the method according to claim 1.

6. A dermo-cosmetic composition in a form of emulsion, wherein the composition is obtained by the method according to claim 2.

7. A dermo-cosmetic composition in a form of simple oil in water emulsion, wherein the composition is obtained by the method according to claim 1.

8. A dermo-cosmetic composition in a form of emulsion, wherein the composition is obtained by the method according to claim 1, and that is does not contain any other emulsifying agent.

9. A use of a composition according to claim 5 for cosmetic care by topical application on a skin, a mucosa and/or a hair.

10. A use of cationised cellulose microfibres with a degree of cationic substitution greater than 0.15, as an emulsifier in a composition according to claim 5.

11. A use of cationised cellulose microfibres according to claim 10, wherein the cellulose microfibres are oat microfibres.

12. The method for preparing a dermo-cosmetic composition according to claim 2, wherein the homogenisation is done by colloidal grinding.

13. The method for preparing a dermo-cosmetic composition according to claim 2, wherein the fat is an oil.

14. The method for preparing a dermo-cosmetic composition according to claim 3, wherein the fat is an oil.

15. The dermo-cosmetic composition in the form of simple oil in water emulsion according to claim 7, wherein the cellulose fibres are oat fibres.

16. The dermo-cosmetic composition in the form of simple oil in water emulsion according to claim 7, wherein the homogenization is done by colloidal grinding.

17. The dermo-cosmetic composition in the form of emulsion according to claim 8, wherein the cellulose fibres are oat fibres.

18. The dermo-cosmetic composition in the form of emulsion according to claim 8, wherein the homogenization is done by colloidal grinding.

19. The use of a composition in a form of emulsion according to claim 9, wherein the composition is obtained by proceeding with cationization in an aqueous phase of cellulosic fibres up to reaching a degree of cationic substitution greater than 0.15, then proceeding with a homogenization of the cationised fibres in the presence of at least one fat up to obtaining a homogenous emulsion, the concentration of the cationised cellulose microfibres being greater than 0.8% by dry weight in relation to the total weight of the composition, and wherein the cellulose fibres are oat fibres.

20. The use of a composition in a form of simple oil in water emulsion according to claim 9, wherein the composition is obtained by proceeding with cationization in an aqueous phase of cellulosic fibres up to reaching a degree of cationic substitution greater than 0.15, then proceeding with a homogenization of the cationised fibres in the presence of at least one fat up to obtaining a homogenous emulsion, the concentration of the cationised cellulose microfibres being greater than 0.8% by dry weight in relation to the total weight of the composition.
